(19) United States
(12) Patent Application Publication
(10) Pub. No.: US 2023/0025286 A1
(43) Pub. Date: Jan. 26, 2023

(54) PHARMACEUTICAL COMPOSITION COMPRISING ELTROMBOPAG BIS(MONOETHANOLAMINE)

(71) Applicant: SYNTHON B.V., Nijmegen (NL)

(72) Inventors: Luis NOGUEIRAS NIETO, Sant Boi de Llobregat (ES); Lisardo ALVAREZ FERNANDEZ, Sant Boi de Llobregat (ES); Rohit KUMAR, Sant Boi de Llobregat (ES)

(21) Appl. No.: 17/782,408

(22) PCT Filed: Dec. 4, 2020
(86) PCT No.: PCT/EP2020/084690

§ 371 Date: Jun. 3, 2022

(30) Foreign Application Priority Data
Dec. 6, 2019 (EP) 19214278.4

(51) Int. Cl. A61K 31/4152; A61K 9/20; A61K 47/12; A61K 47/32

(52) U.S. Cl. CPC A61K 31/4152; A61K 9/2018; A61K 47/12; A61K 47/32

(57) ABSTRACT

The present invention relates to a film-coated swallowable tablet comprising eltrombopag bis(monoethanolamine) and pharmaceutically acceptable excipients comprising maltose and isomalt as diluents. The invention further relates to the use of said tablet as a medicament, particularly in the treatment of immune thrombocytopenia (ITP), thrombocytopenia inpatients with chronic hepatitis C virus (HCV) and severe aplastic anaemia (SAA).

### BACKGROUND OF THE PRESENT INVENTION

Eltrombopag bis(monoethanolamine), chemically 3′-{(2Z)-2-[1-(3,4-dimethylphenyl)-3-methyl-5-oxo-1,5-dihydro-4H-pyrazol-4-ylidene]hydrazino}-2′-hydroxy-3-biphenylcarboxylic acid bis(monoethanolamine) of formula (I),

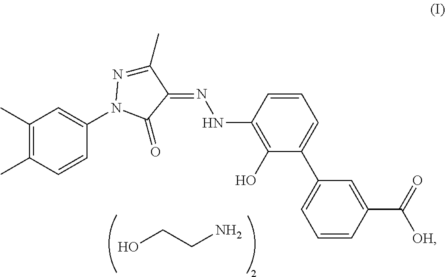

is a pharmaceutically active compound. It is used for the treatment of immune thrombocytopenia (ITP), thrombocytopenia in patients with chronic hepatitis C virus (HCV) and severe aplastic anaemia (SAA).

Eltrombopag bis(monoethanolamine), also known as eltrombopag olamine, is marketed by Novartis under the brand names Revolade® in Europe and Promacta® in the US and is disclosed in WO2001089457. Revolade® and Promacta® are supplied as immediate release film-coated tablets. Revolade® is available in three strengths: 25, 50 and 75 mg. Promacta® is available in five strengths: 12.5, 25, 50, 75 and 100 mg. The tablet composition of the different strengths is not dose proportional.

Eltrombopag bis(monoethanolamine) exhibits low solubility and medium permeability. WO2008136843 discloses the pharmaceutical composition of the Revolade®/Promacta® tablets. The Revolade®/Promacta® tablet formulation comprises, besides eltrombopag bis(monoethanolamine), the diluents microcrystalline cellulose and mannitol. According to WO2008136843, the use of diluents comprising reducing sugars or coordinating metals should be avoided because of the fact that these compounds may undergo a Maillard reaction or form an insoluble metal complex with eltrombopag bis(monoethanolamine).

WO2015029074 discloses compositions comprising eltrombopag olamine and more than 12% by weight based on the total weight of the composition of a disintegrant.

CN107693515 discloses pharmaceutical compositions comprising eltrombopag, or a salt thereof, and at least one alkalizing agent comprising a monovalent acid salt of a weak acid or a monovalent metal hydroxide. The dissolution rate is improved by using the alkalizing agent.

WO2018197088 discloses pharmaceutical tablet compositions comprising eltrombopag olamine and one or more reducing sugars selected from the group consisting of lactose, maltose, glucose, arabinose and fructose, wherein eltrombopag olamine is present in the intragranular phase and the reducing sugar in the extragranular phase or vice versa.

WO2019086725 discloses compositions comprising eltrombopag olamine, one or more reducing sugars selected from the group consisting of lactose, maltose, glucose, arabinose and fructose, and one or more semisynthetic and/or synthetic polymer binder agent. The composition is obtained by granulation. Eltrombopag olamine, the reducing sugar(s) and binder agent(s) are all present in the intragranular or extragranular composition.

It would be desirable to have a tablet composition comprising eltrombopag bis(monoethanolamine) that exhibits adequate dissolution and disintegration. The composition should exhibit excellent stability, be suitable for production on commercial scale and bioequivalent to Revolade®/Promacta®. Furthermore, it would be advantageous if the different tablet strengths can be formulated in a dose proportional manner to require just one single blend.

### BRIEF DESCRIPTION OF THE PRESENT INVENTION

The present invention provides a film-coated swallowable tablet comprising eltrombopag bis(monoethanolamine) and pharmaceutically acceptable excipients comprising maltose and isomalt as diluents.

It also provides a process to prepare the tablet comprising wet granulation.

Said pharmaceutical composition may be used as a medicament, particularly in the treatment of immune thrombocytopenia (ITP), thrombocytopenia in patients with chronic hepatitis C virus (HCV) and severe aplastic anaemia (SAA).

### DETAILED DESCRIPTION OF THE PRESENT INVENTION

The film-coated tablet Revolade®/Promacta® comprises, besides eltrombopag bis(monoethanolamine), the diluents microcrystalline cellulose and mannitol. According to WO2008136843, the use of diluents comprising coordinating metals should be avoided because of the fact that these metals may form an insoluble metal complex with eltrombopag bis(monoethanolamine). Moreover, the use of diluents comprising reducing sugars should be avoided because these compounds may undergo a Maillard reaction with eltrombopag bis(monoethanolamine).

The present invention provides a film-coated swallowable tablet comprising eltrombopag bis(monoethanolamine) and pharmaceutically acceptable excipients comprising maltose and isomalt as diluents. It was surprisingly found by the present inventors that, although maltose is a reducing sugar, this diluent can be applied in the tablet comprising eltrombopag bis(monoethanolamine) without giving rise to issues due to the occurrence of the Maillard reaction. In fact, the use of maltose in combination with isomalt in the tablet comprising eltrombopag bis(monoethanolamine) provides a tablet with excellent properties. The tablet exhibits very good stability and is bioequivalent to Revolade®/Promacta®. Moreover, the different tablet strengths are formulated in a dose proportional manner having the advantage that just one single blend is required to make all strengths.

Taking into account the teaching of WO2008136843, the choice for maltose as diluent is certainly not obvious. In the tablet of the present invention, maltose is present in an amount of 10 to 40% by weight based on the total weight of the tablet. More preferably, the amount of maltose in the tablet is in the range of 15 to 30% by weight based on the total weight of the tablet. Most preferably, the amount of maltose in the tablet is in the range of 20 to 25% by weight based on the total weight of the tablet. Preferably, maltose is present in the tablet of the present invention in the extragranular phase of the tablet composition.

Isomalt is used in combination with maltose in the tablet of the present invention. By using just maltose, the compression performance of the tablet is not sufficient. By using a combination of maltose and isomalt, the desired tablet hardness can already be achieved by applying relatively low compression forces. Generally, isomalt comprises a mixture of 6-O-α-D-glucopyranosyl-D-sorbitol (1,6-GPS) and 1-O-α-D-glucopyranosyl-D-mannitol dehydrate (1,1-GPM). 1,6-GPS is more soluble than 1,1-GPM. By shifting the ratio of the two components, the solubility and crystal water content can be adjusted. Preferably, the isomalt used in the tablet of the present invention is a grade comprising 1,6-GPS and 1,1-GPM in a ratio of 1:1. A typical and preferred example of a commercially available grade of isomalt, wherein 1,6-GPS and 1,1-GPM are present in a 1:1 ratio, is galenIQ™ 720. Preferably, the isomalt used in the present invention has a particle size distribution D₉₀ ranging from 300 to 400 μm. Most preferably, the particle size distribution D₉₀ ranges from 325 to 375 μm.

Additional advantage of isomalt is its intense and well-balanced sweet taste, which is very close to sucrose. It does not have any significant off-taste or aftertaste.

In the tablet of the present invention, isomalt is present in an amount of 10 to 40% by weight based on the total the weight of the tablet. More preferably, the amount of isomalt in the tablet is in the range of 15 to 30% by weight based on the total weight of the tablet. Most preferably, the amount of isomalt in the tablet is in the range of 20 to 25% by weight based on the total weight of the tablet.

Isomalt is, like the maltose, preferably present in the extragranular phase of the tablet composition.

The tablet in accordance with the present invention may comprise, besides eltrombopag bis(monoethanolamine), maltose and isomalt, an acidulant. An acidulant has a retarding effect on the dissolution of the tablet. Additional benefit of the use of an acidulant is that by creating an acidic environment, the likelihood of occurrence of the Maillard reaction between eltrombopag bis(monoethanolamine) and the reducing sugar maltose is reduced. The acidulant is present in the intragranular phase and is preferably added in an aqueous solution. In solution, the acidulant has the optimal distribution and impact on the solubility of the active pharmaceutical ingredient.

Acidulants that can be used in accordance with the present invention are citric acid, malic acid or tartaric acid. A particularly preferred acidulant is citric acid.

In the tablet of the present invention, citric acid is present in an amount of 0.2 to 1.5% by weight based on the total the weight of the tablet. More preferably, the amount of citric acid in the tablet is in the range of 0.25 to 0.75% by weight based on the total weight of the tablet. Most preferably, the amount of citric acid in the tablet is in the range of 0.4 to 0.5% by weight based on the total weight of the tablet. By using this amount of citric acid, the optimal effect on the dissolution profile is achieved.

The tablet of the present invention is preferably prepared by a process comprising wet granulation. The granulation process applied is simple and cost effective and includes a standard wet granulation technique.

The wet granulation process is performed with a granulation solvent selected from the group consisting of water, acetone, ethanol, isopropanol or a mixture thereof. The most preferred solvent to be used in accordance with the present invention is water.

At least 70% of the granules obtained by the process of wet granulation have a particle size between 90 and 355 μm. At least 45% of the granules have a particle size from 125 to 250 μm. Furthermore, maximum 15% of the granule particles are bigger than 355 μm and maximum 15% of the granules are smaller than 90 μm. By using granules with these particular particle size specifications, both good flow properties and compression performance are achieved. By limiting the amount of fines, sticking issues are prevented, while by limiting the amount of big granules, a good content uniformity is achieved.

The film-coated swallowable tablet comprising eltrombopag bis(monoethanolamine), maltose, isomalt and optionally an acidulant, further comprises pharmaceutically acceptable excipients. The excipients to be used in accordance with the present invention are well-known and are those excipients which are conventionally used by the person skilled in the art in pharmaceutical compositions. The excipients are selected from one or more diluents, binders, disintegrants, glidants or lubricants.

The diluent to be used in accordance with the present invention, in combination with maltose and isomalt, may be any diluent known to a person of ordinary skill in the art. Preferably, these diluents are inorganic diluents, polysaccharides, mono- or disaccharides or sugar alcohols. Microcrystalline cellulose and mannitol are particularly preferred diluents.

The binder to be used in accordance with the present invention may be any binder known to a person of ordinary skill in the art. Suitable binders are selected from the group consisting of celluloses, starch, polyethylene glycol (PEG), sodium carboxymethylcellulose and polyvinyl pyrrolidone (PVP). PVP, also known as povidone, is a particularly preferred binder. The amount of binder in the film-coated swallowable tablet is between 1 and 5% by weight based on the total weight of the tablet. Preferably, the amount of binder in the tablet is between 1.5 and 4% by weight based on the total weight of the tablet. More preferably, the amount of binder in the tablet is between 2 and 3% by weight based on the total weight of the tablet.

The disintegrant to be used in accordance with the present invention may be any disintegrant known to a person of ordinary skill in the art. Suitable disintegrants to be used in accordance with the present invention are selected from the group consisting of croscarmellose sodium, crospovidone or sodium starch glycolate. Sodium starch glycolate is a particularly preferred disintegrant. The amount of disintegrant in the tablet is between 5 and 15% by weight based on the total weight of the tablet. Preferably, the amount of disintegrant in the tablet is between 8 and 12% by weight based on the total weight of the tablet.

The glidant to be used in accordance with the present invention may be any glidant known to a person of ordinary skill in the art. Colloidal silicon dioxide and talc are particularly preferred glidants.

The lubricant to be used in accordance with the present invention may be any lubricant known to a person of ordinary skill in the art. Magnesium stearate is a particularly preferred lubricant. The amount of lubricant in the film-coated swallowable tablet is between 0.5 and 2% by weight based on the total weight of the tablet.

The tablet of the present invention is coated by a film-coat. The coating serves cosmetic purposes. The coating material typically has no influence on the release rate, except of an inherent short initial delay in dissolution due to the time necessary to dissolve the coating. The coating may be selected from amongst one or more of those suitable coating materials known in the art.

The coating may be performed by applying one or more film forming polymers, with or without other pharmaceutically inert excipients, as a solution/suspension. Coating is done using any conventional coating technique known in the art, such as spray coating in a conventional coating pan or fluidized bed processor; or dip coating.

The film-coated swallowable tablet of the present invention exhibits excellent long term stability. Moreover, the tablet of the present invention is very suitable for production on commercial scale making use of equipment and techniques commonly used in industry.

The film-coated swallowable tablets of the present invention are preferably packed in blister pack material. The blister pack material to be used in accordance with the present invention may be any blister pack material known to a person of ordinary skill in the art. Suitable blister pack materials to be used in accordance with the present invention are PVC/Alu, PVDC/PVC/Alu and Alu/Alu. A particularly preferred blister pack material to be used in accordance with the present invention is Alu/Alu.

The tablet composition according to the present invention displays dissolution behavior typical for immediate-release formulations.

The tablet composition in accordance with the present invention may be used as a medicament. The composition typically may be used in the treatment of immune thrombocytopenia (ITP), thrombocytopenia in patients with chronic hepatitis C virus (HCV) and severe aplastic anaemia (SAA).

The following examples are intended to illustrate the scope of the present invention but not to limit it thereto.

### EXAMPLES

### Example 1: Film-Coated Tablet Comprising Eltrombopag Bis(Monoethanolamine), Maltose and Isomalt

The film-coated tablet comprising eltrombopag bis(monoethanolamine), maltose and isomalt has the composition as given in table 1.

TABLE 1

| | Tablet composition | |
|---|---|---|
| Component | mg/tablet | % |

Intragranular components

| | | |
|---|---|---|
| Eltrombopag bis(monoethanolamine) | 95.70 | 19.94 |
| Microcrystalline cellulose | 30.72 | 6.40 |
| Mannitol | 63.36 | 13.20 |
| Povidone | 14.40 | 3.00 |
| Purified water | q.s. | q.s. |

Extragranular components

| | | |
|---|---|---|
| Isomalt | 111.51 | 23.23 |
| Maltose | 111.51 | 23.23 |
| Sodium starch glycolate | 48.00 | 10.00 |
| Magnesium stearate | 4.80 | 1.00 |
| Total core tablet weight | 480.00 | 100.00 |
| Opadry coating | 19.20 | 4.00 |
| Purified water | q.s. | q.s. |
| Total coated tablet weight | 499.20 | 104.00 |

The intragranular components were sieved to de-agglomerate and added to a high shear granulator bowl. Water was added until an adequate granule appearance was obtained. The obtained granulate was dried and sieved. The extragranular isomalt, maltose and sodium starch glycolate were sieved to de-agglomerate and the excipients were mixed with the sieved granules in a diffusion mixer. The magnesium stearate was mixed, added to the diffusion mixer and the resulting mixture was mixed. The homogeneous blend was compressed on a rotary tablet press using appropriate punches.

The tablets obtained were packed in alu/alu blisters.

### Example 2: Film-Coated Tablet Comprising Eltrombopag Bis(Monoethanolamine), Maltose, Isomalt and Citric Acid

The film-coated tablet comprising eltrombopag bis(monoethanolamine), maltose, isomalt and citric acid has the composition as given in table 2.

TABLE 2

| | Tablet composition | |
|---|---|---|
| Component | mg/tablet | % |

Intragranular components

| | | |
|---|---|---|
| Eltrombopag bis(monoethanolamine) | 95.70 | 19.94 |
| Microcrystalline cellulose | 30.72 | 6.40 |
| Mannitol | 63.36 | 13.20 |
| Povidone | 14.40 | 3.00 |
| Anhydrous citric acid | 2.40 | 0.50 |
| Purified water | q.s. | q.s. |

Extragranular components

| | | |
|---|---|---|
| Isomalt | 110.31 | 22.98 |
| Maltose | 110.31 | 22.98 |
| Sodium starch glycolate | 48.00 | 10.00 |
| Magnesium stearate | 4.80 | 1.00 |
| Total core tablet weight | 480.00 | 100.00 |
| Opadry coating | 19.20 | 4.00 |
| Purified water | q.s. | q.s. |
| Total coated tablet weight | 499.20 | 104.00 |

The intragranular components eltrombopag bis(monoethanolamine), microcrystalline cellulose, mannitol and povidone were sieved to de-agglomerate and added to a high shear granulator bowl. The components were mixed. A solution of anhydrous citric acid in water was added. Additional water was added until an adequate granule appearance was obtained. The obtained granulate was dried and sieved. The extragranular isomalt, maltose and sodium starch glycolate were sieved to de-agglomerate and the excipients were mixed with the sieved granules in a diffusion mixer. The magnesium stearate was mixed, added to the diffusion mixer and the resulting mixture was mixed. The homogeneous blend was compressed on a rotary tablet press using appropriate punches.

The tablets obtained were packed in alu/alu blisters.

1. A film-coated swallowable tablet comprising eltrombopag bis(monoethanolamine) and pharmaceutically acceptable excipients comprising maltose and isomalt as diluents.

2. The tablet according to claim 1, wherein maltose is present in an amount of 10 to 40% by weight based on the total weight of the tablet.

3. The tablet according to claim 1, wherein the isomalt is present in an amount of 10 to 40% by weight based on the total weight of the tablet.

4. The tablet according to claim 1, wherein the isomalt has a particle size distribution D₉₀ ranging from 300 to 400 μm.

5. The tablet according to claim 1, wherein maltose and isomalt are present in an extragranular phase.

6. The tablet according to claim 1, further comprising an acidulant as excipient.

7. The tablet according to claim 6, wherein the acidulant is present in an intragranular phase.

8. The tablet according to claim 6, wherein the acidulant is citric acid.

9. The tablet according to claim 8, wherein citric acid is present in an amount of 0.2 to 1.5% by weight based on the total weight of the tablet.

10. The tablet according to claim 1, wherein the pharmaceutically acceptable excipients are selected from one or more diluents, binders, disintegrants, glidants or lubricants.

11. The tablet according to claim 10, wherein the amount of binder is between 1 and 5% by weight based on the total weight of the tablet.

12. The tablet according to claim 10, wherein the binder is polyvinylpyrrolidone (PVP).

13. A process to prepare the tablet according to claim 1 comprising wet granulation.

14. The process according to claim 13, wherein at least 70% of the obtained granules have a particle size between 90 and 355 μm.

15. (canceled)

16. A method of treating immune thrombocytopenia (ITP), thrombocytopenia in patients with chronic hepatitis C virus (HCV) and/or severe aplastic anaemia (SAA) in a patient, which comprises administering the tablet according to claim 1 to a patient in need thereof.

17. The process according to claim 13, which comprises:
(i) wet granulating eltrombopag bis(monoethanolamine), a binder, and optionally additional pharmaceutically acceptable excipients to form a granulate;
(ii) combining said granulate with maltose, isomalt, and optionally additional pharmaceutically acceptable excipients to form a tablet blend;
(iii) tableting said tablet blend to form a tablet; and
(iv) film coating said tablet.

18. The tablet according to claim 1, wherein said eltrombopag bis(monoethanolamine) is present in an intragranular phase and said maltose and isomalt are present in an extragranular phase; and wherein said maltose is present in an amount of 10 to 40% by weight based on the total weight of the tablet, and said isomalt is present in an amount of 10 to 40% by weight based on the total weight of the tablet.

19. The tablet according to claim 18, wherein said intragranular phase further comprises a binder.

20. The tablet according to claim 19, wherein said intragranular phase further comprises an acidulant as an excipient.

21. The tablet according to claim 20, wherein said intragranular phase further comprises a diluent and said extragranular phase further comprises a disintegrant.
